(19) United States
(12) Patent Application Publication
(10) Pub. No.: US 2026/0315519 A1
(43) Pub. Date: Oct. 15, 2026

(54) COMPOSITIONS COMPRISING METHYL CYCLODEXTRINS FOR THE TREATMENT AND/OR PREVENTION OF HEPATIC STEATOSIS

(71) Applicant: ROQUETTE FRERES, LESTREM (FR)

(72) Inventors: Daniel WILS, MORBECQUE (FR); Caroline PERREAU, LA COUTURE (FR)

(21) Appl. No.: 19/719,586

(22) Filed: Jun. 25, 2026

Related U.S. Application Data
(63) Continuation of application No. 18/251,358, filed on Nov. 4, 2021.

(30) Foreign Application Priority Data
Nov. 5, 2020 (FR) 2011361

(51) Int. Cl. A61K 31/724; A61P 1/16

(52) U.S. Cl. CPC A61K 31/724; A61P 1/16

(57) ABSTRACT

The present invention relates to a novel use of a pharmaceutical composition comprising at least one methyl cyclodextrin in the treatment and/or prevention of hepatic steatosis and related diseases. The invention also relates to the use of methyl cyclodextrin to reduce lipid accumulation in the liver.

### CROSS-REFERENCE TO RELATED APPLICATION

This application is a Continuation of U.S. patent application Ser. No. 18/251,358, filed on May 1, 2023, which is a national stage application of International Application No. PCT/EP2021/025430 filed on Nov. 4, 2021, which claims benefit of French Patent Application No. 2011361, filed on Nov. 5, 2020. Each of prior mentioned applications are hereby incorporated by reference in their entireties.

### TECHNICAL FIELD

The present invention relates to pharmaceutical compositions for reducing the storage of lipids in the liver in an individual. The present invention more specifically relates to the use of a pharmaceutical composition in the treatment and/or prevention of hepatic steatosis and diseases or conditions associated with hepatic steatosis.

### TECHNOLOGICAL BACKGROUND OF THE INVENTION

Non-alcoholic steatopathy (non-alcoholic fatty liver disease or NAFLD) is characterized by abnormal accumulation of intrahepatic fat in the absence of excessive consumption of alcohol.

NAFLD encompasses a wide spectrum of hepatic pathologies in which two major entities can be distinguished: steatosis, either isolated or accompanied by minimal lobular inflammation (non-alcoholic fatty liver or NAFL) and non-alcoholic steatohepatitis (or NASH). NASH is defined by the presence of steatosis with lobular inflammation and hepatocytes ballooning. It corresponds to the aggressive form of the disease which promotes the accumulation of fibrosis in the hepatic parenchyma, progressing to cirrhosis and its complications (hepatic failure, ascites, variceal rupture, hepatocarcinoma).

The social and medical care impact of NAFLD and NASH is very large, and constantly growing. It is estimated that approximately 30% of the United States population suffers from NAFLD. The prevalence of NASH is 8% in the United States.

Several genetic factors predisposing to NAFLD and its severity have been identified. NAFLD operates in a dysmetabolic and insulin resistance context. The accumulation of the criteria of the metabolic syndrome (waist size, arterial pressure, fasting glucose level, triglycerides, HDL-cholesterol) and the degree of insulin resistance are associated with an increase in the prevalence of NAFLD and its severity (NASH, fibrosis).

Environmental factors (poorly balanced diet, lack of sports activity) are also significant risk factors.

NAFLD is generally a slowly progressing disease, but its natural history is still poorly known. NASH represents the aggressive form of the disease: compared to patients with merely NAFL, patients with NASH have a greater rate of progression of the fibrosis, tending more toward cirrhosis, developing more hepatic complications and having higher mortality.

The mechanisms involved in the development of this disease involve an accumulation of lipids in the liver, followed by inflammation and a scarring process. In the most advanced stages (cirrhosis), the hepatic tissue is gradually replaced with scar tissue.

The treatments envisaged to date are insufficient, and most often act downstream of the pathological process. Mention may be made, for example, of caspase or ASK1 inhibitors, which act on the cell death process that leads to inflammation.

Finally, there is still no medication on the drug market capable of acting upstream, in order to limit the accumulation of lipids in the liver. In addition, there is a constant need for drugs capable of controlling hepatic steatosis and diseases or conditions associated with hepatic steatosis.

### SUMMARY

It is to the Applicant's credit to have discovered that pharmaceutical compositions based on methyl cyclodextrins make it possible to reduce the storage of lipids in the liver, and thus be useful for the treatment and/or prevention of hepatic steatosis.

Thus, the present invention relates to a pharmaceutical composition comprising at least one methyl cyclodextrin for use in the treatment and/or prevention of hepatic steatosis and diseases associated with hepatic steatosis.

The invention also relates to the use of a methyl cyclodextrin for the manufacture of a medicament for use in the treatment and/or prevention of hepatic steatosis.

In addition, the present invention provides a method for treating and/or preventing hepatic steatosis comprising administering to a patient a therapeutically effective amount of methyl cyclodextrin.

### DETAILED DESCRIPTION OF THE INVENTION

The inventors have identified a new use of a pharmaceutical composition comprising at least one methyl cyclodextrin in the treatment and/or prevention of hepatic steatosis and diseases associated with hepatic steatosis

Within the meaning of the present invention, the term “hepatic steatosis” covers both alcoholic steatoses, linked to excessive consumption of alcohol, and non-alcoholic steatoses. Preferably, it involves non-alcoholic steatosis.

The term “non-alcoholic steatosis” encompasses all stages of evolution of a pathology wherein the liver is afflicted and characterized by excessive accumulation of lipids. It may be non-alcoholic hepatic steatosis (NAFLD, for “non-alcoholic fatty liver disease”) and non-alcoholic steato-hepatitis (NASH).

The invention also relates to the treatment and/or prevention of diseases or conditions associated with hepatic steatosis, such as acute or chronic liver inflammation, hepatic fibrosis, abdominal obesity, hepatic failure and cirrhosis.

In one embodiment of the invention, the disease associated with hepatic steatosis is not diabetes.

Cyclodextrins are cyclic oligosaccharides derived from the enzymatic degradation of starch. The three most common natural cyclodextrins consist of 6, 7, or 8 α-D-glucopyranose units in the chair configuration linked together by α-1,4 bonds. They are more commonly called α, β, or γcyclodextrin, respectively. Their three-dimensional structure occurs in the form of a truncated cone, outside of which are the hydroxyl groups representing the highly hydrophilic part of cyclodextrins. The interior of the cone or cavity of cyclodextrins consists of the hydrogen atoms carried by the C3 and C5 carbons as well as by the oxygen atoms participating in the glycosidic bond, thus conferring a non-polar character to them. Cyclodextrins having a hydrophilic outer part and a hydrophobic cavity are generally used for their ability to encapsulate hydrophobic compounds, and, thus, for their role as protector and solubilizer of hydrophobic active ingredients. They are typically found in the food industry, but also in medicinal form where they are used as excipients in pharmaceutical formulations administered orally or in cosmetic formulations administered topically.

In order to improve the aqueous solubility of natural cyclodextrins, many derivatives were synthesized by grafting different groups onto the hydroxyl functions. To wit, the glucopyranose units of cyclodextrins each comprise 3 reactive hydroxyl groups, which are carried on the C2, C3 and C6 carbons.

Example derivatives include hydroxypropyl-cyclodextrins, methyl cyclodextrins and “sulfated” derivatives of cyclodextrin.

The Applicant has surprisingly demonstrated that methyl cyclodextrin could also be used in the treatment and/or prevention of hepatic steatosis. Even more surprisingly, she has discovered that methyl cyclodextrin, and more particularly a methyl cyclodextrin having a molar substitution value of between 0.05 and 1.5, was even more effective than other cyclodextrin derivatives to reduce the accumulation of lipids in the liver.

In addition, the Applicant has demonstrated that a methyl cyclodextrin having a molar substitution value of between 0.05 and 1.5 was capable of promoting the increase in the removal of cholesterol.

The term “molar substitution value (MS)” is understood to mean the number of hydroxyls substituted, in particular by a methyl group, for each glucopyranose unit. Note that the molar substitution value (MS) is different from the molecular degree of substitution (DS) that corresponds to the number of hydroxyls substituted by a methyl group, per molecule of cyclodextrin and that thus takes into account the number of glucopyranose units constituting the methyl cyclodextrin.

The MS can be determined in the present invention by Proton Nuclear Magnetic Resonance (NMR), or by mass spectrometry (electrospray ionization mass spectrometry (ESI-MS) or matrix-assisted laser desorption/ionization mass spectrometry (MALDI-MS)). Although these techniques are well-known to a person skilled in the art, optimal conditions for determining the methyl cyclodextrins according to the invention are in particular well described in the referenced thesis of Romain JACQUET: “Hydrophilic cyclodextrins: characterization and study of their enantioselective and complexing properties. Use of liquid chromatography and mass spectrometry”. Thesis in Chemistry and Physicochemistry of Compounds of Biological Interest. University of Orléans, 2006, available at: http://tel.archives-ouvertes.fr/docs/00/18/55/42/PDF/jacquet.pdf (accessed on 27.11.2013). “, in particular Chapter 2, Part B (pages 59-83).

Preferably, the MS is determined by NMR, according to the following method: the measurements are carried out at 25° C. on a DPX 250 MHZ Advance-type apparatus (Bruker, Rheinstetten, Germany). Calibration is carried out with the D20 signal. Samples of methyl cyclodextrin according to the invention, and of native (that is to say non-methylated) cyclodextrin are prepared at a concentration of 5 mg in 0.75 mL of D20. The solutions are evaporated to dryness under a stream of nitrogen and then reconstituted in 0.75 mL of D20. This operation is repeated twice to ensure a total exchange of protons of the hydroxyl functions.

It should be noted that the methyl cyclodextrin used according to the invention, although it may correspond to a pure product, generally corresponds to a mixture of methyl cyclodextrins of different structures. This is the case for example of the product KLEPTOSE® CRYSMEB owned by the Applicant, which in particular has the physical/chemical properties as determined in the aforementioned Romain JACQUET thesis, in particular in chapter 2, part B (pages 59 to 83).

As a result, the measured MS in this case is an average of the substitutions that take place on all the glucopyranose units of the whole mixture of methyl cyclodextrins.

This mixture may in particular contain residual native, that is to say non-methylated, cyclodextrins, but generally in negligible amounts, in particular less than 1% by dry weight relative to the total dry weight of the methyl cyclodextrin, preferably less than 0.5%, even more preferentially less than 0.1%.

In the context of the invention, the compositions comprise at least one methyl cyclodextrin having a molar substitution value of between 0.05 and 1.5. Advantageously, the methyl cyclodextrin has an MS comprised between 0.1 and 1.4, preferentially between 0.1 and 1.3, preferentially between 0.2 and 1.2, preferentially between 0.3 and 1.1, preferentially between 0.3 and 1, preferentially between 0.5 and 0.9, preferentially between 0.6 and 0.8, for example 0.7, in particular 0.67. For example, the methyl cyclodextrin may have a MS of between 0.10 and 1.40, between 0.10 and 1.30, between 0.10 and 1.20, between 0.15 and 1.40, between 0.15 and 1.30, between 0.15 and 1.20, between 0.20 and 1.40, between 0.20 and 1.30, between 0.20 and 1.20, between 0.20 and 1.10, between 0.25 and 1.40, between 0.25 and 1.30, between 0.25 and 1.20, between 0.25 and 1.10, between 0.15 and 0.90, between 0.15 and 0.80, between 0.25 and 1.00, between 0.25 and 0.90, between 0.25 and 0.80, between 0.30 and 1.40, between 0.30 and 1.30, between 0.30 and 1.20, between 0.30 and 1.00, between 0.50 and 0.90, between 0.60 and 0.80.

Preferably, at least 50% of the methyl groups of the methyl cyclodextrin used in the context of the present invention are located at the hydroxyl carried by the C2 carbon of the glucopyranose unit, preferably between 60 and 80%, typically on the order of 75%.

At the same time, the other methyl groups are generally located mainly at the hydroxyl carried by the C3 and/or C6 carbon of the glucopyranose unit.

The person skilled in the art knows how to determine the distribution of methyl groups on the hydroxyls of the glucopyranose unit of methyl cyclodextrin, for example by NMR.

Advantageously, the methyl cyclodextrin used in the context of the present invention comprises 7 α-D-glucopyranose units. It is therefore a methyl-β-cyclodextrin.

In a particular embodiment, methyl cyclodextrin is a methyl-β-cyclodextrin and has an MS of between 0.05 and 1.5, preferentially between 0.1 and 1.4, preferentially between 0.1 and 1.3, preferentially between 0.2 and 1.2, preferentially between 0.3 and 1.1, preferentially between 0.4 and 1, preferentially between 0.5 and 0.9, preferentially between 0.6 and 0.8, for example 0.7, in particular 0.67. For example, the methyl cyclodextrin may have a MS of between 0.10 and 1.40, between 0.10 and 1.30, between 0.10 and 1.20, between 0.15 and 1.40, between 0.15 and 1.30, between 0.15 and 1.20, between 0.20 and 1.40, between 0.20 and 1.30, between 0.20 and 1.20, between 0.20 and 1.10, between 0.25 and 1.40, between 0.25 and 1.30, between 0.25 and 1.20, between 0.25 and 1.10, between 0.25 and 1.00, between 0.25 and 0.90, between 0.25 and 0.80, between 0.30 and 1.40, between 0.30 and 1.30, between 0.30 and 1.20, between 0.30 and 1.00, between 0.50 and 0.90, between 0.60 and 0.80.

The methyl cyclodextrin may be substituted on the hydroxyl carried by the C2 carbon of the glucopyranose units, or by the C3 and/or C6 carbons of the glucopyranose units, or by a combination of the C2, C3 and/or C6 carbons, preferably C2 and C6 carbons of the glucopyranose units.

In another particular embodiment, the methyl cyclodextrin is a methyl cyclodextrin, preferably a methyl-β-cyclodextrin, of which at least 50% of the methyl groups are located at the hydroxyl carried by the C2 carbon of the glucopyranose unit, preferentially between 60 and 80%, typically of the order of 75%, and has an MS of between 0.05 and 1.5, preferentially between 0.1 and 1.4, preferentially between 0.1 and 1.3, preferentially between 0.2 and 1.2, preferentially between 0.3 and 1.1, preferentially between 0.4 and 1, preferentially between 0.5 and 0.9, preferentially between 0.6 and 0.8, for example 0.7, in particular 0.67. For example, the methyl cyclodextrin may have a MS of between 0.10 and 1.40, between 0.10 and 1.30, between 0.10 and 1.20, between 0.15 and 1.40, between 0.15 and 1.30, between 0.15 and 1.20, between 0.20 and 1.40, between 0.20 and 1.30, between 0.20 and 1.20, between 0.20 and 1.10, between 0.25 and 1.40, between 0.25 and 1.30, between 0.25 and 1.20, between 0.25 and 1.10, between 0.25 and 1.00, between 0.25 and 0.90, between 0.25 and 0.80, between 0.30 and 1.40, between 0.30 and 1.30, between 0.30 and 1.20, between 0.30 and 1.00, between 0.50 and 0.90, between 0.60 and 0.80.

In a preferred embodiment, the methyl cyclodextrin composition comprises one or more methyl-β-cyclodextrins chosen from the group consisting of methyl-β-cyclodextrins substituted on the hydroxyl carried by C2 carbon of the glucopyranose units, methyl-β-cyclodextrins substituted on the hydroxyl carried by carbon C3 and/or C6 of the glucopyranose units, methyl-β-cyclodextrins substituted on the hydroxyl carried by carbons C2, C3 and/or C6, preferably C2 and C6, glucopyranose units and having a MS of between 0.05 and 1.5, preferentially between 0.1 and 1.4, preferentially between 0.1 and 1.3, preferentially between 0.2 and 1.2, preferentially between 0.3 and 1.1, preferentially between 0.4 and 1, preferentially between 0.5 and 0.9, preferentially between 0.6 and 0.8, for example 0.7, particularly 0.67. For example, the methyl cyclodextrin may have a MS of between 0.10 and 1.40, between 0.10 and 1.30, between 0.10 and 1.20, between 0.15 and 1.40, between 0.15 and 1.30, between 0.15 and 1.20, between 0.20 and 1.40, between 0.20 and 1.30, between 0.20 and 1.20, between 0.20 and 1.10, between 0.25 and 1.40, between 0.25 and 1.30, between 0.25 and 1.20, between 0.25 and 1.10, between 0.25 and 1.00, between 0.25 and 0.90, between 0.25 and 0.80, between 0.30 and 1.40, between 0.30 and 1.30, between 0.30 and 1.20, between 0.30 and 1.00, between 0.50 and 0.90, between 0.60 and 0.80. Preferably, the methyl cyclodextrin composition comprises at least 50, 60, or 75% methyl substituted on the hydroxyl carried by the C2 carbon of the glucopyranose units.

As mentioned above, the methyl cyclodextrin according to the invention can be a mixture. The mass spectrometry analysis of the KLEPTOSER CRYSMEB product, which is a methyl-β-cyclodextrin, reveals in particular that it is a polydispersed product, comprising seven majority methyl cyclodextrin groups, which are distinguished by their DS. This DS, which in theory may vary from 0 to 21 for a methyl-β-cyclodextrin, varies from 2 to 8 in the KLEPTOSER CRYSMEB product.

Advantageously, the compositions of the invention comprise a methyl cyclodextrin mixture comprising at least 50, 60, 70, 80 or 90% methyl cyclodextrins having a MS of between 0.2 and 1.2. Preferably at least 40, 50, 60, 70, 80 or 90% of methyl cyclodextrins have a MS of between 0.3 and 1.1. Preferably at least 30, 40, 50, 60, 70, 80 or 90% of methyl cyclodextrins have a MS of between 0.5 and 0.9. Even more preferentially, at least 25, 30, 40, 50, 60, 70, 80 or 90% of methyl cyclodextrins have a MS of between 0.6 and 0.8.

The methyl cyclodextrin compositions may optionally be prepared by adding different methyl cyclodextrins having MSs defined to obtain compositions as defined in the present invention or they may be obtained as a result of the synthesis thereof.

Thus, in another particular embodiment, the methyl cyclodextrin composition, preferably of methyl-β-cyclodextrins, has the substitution profile, expressed in molar percentages, corresponding to:
0 to 5% of methyl-β-cyclodextrins comprise 2 methyl groups (DS of 2);
5 to 15% of methyl-β-cyclodextrins comprise 3 methyl groups (DS of 3);
20 to 25% of methyl-β-cyclodextrins comprise 4 methyl groups (DS of 4);
25 to 40% of methyl-β-cyclodextrins comprise 5 methyl groups (DS of 5);
15 to 25% of methyl-β-cyclodextrins comprise 6 methyl groups (DS of 6);
5 to 15% of methyl-β-cyclodextrins comprise 7 methyl groups (DS of 7);
0 to 5% of methyl-β-cyclodextrins comprise 8 methyl groups (DS of 8).

the total sum being generally of the order of 100%, although the composition can optionally contain traces of methyl cyclodextrins of different DS, as well as traces of native, that is non-methylated, cyclodextrin.

The substitution profile can be determined by any technique known to the person skilled in the art, for example by ESI-MS or MALDI-TOF-MS. The optimal conditions for determining the substitution profile by these two methods are in particular deeply discussed in the aforementioned thesis by Romain JACQUET, in chapter 2, part B, points II.3 and II.2 (page 67 to 82) and in Appendix II.

In a preferred embodiment, the composition of methyl cyclodextrins, preferably of methyl-β-cyclodextrins, is such that at least 50% of the methyl groups are located at the hydroxyl carried by the C2 carbon of the glucopyranose units, preferably between 60 and 80%, typically of the order of 75%, and which has the substitution profile, expressed in molar percentages, according to:
0 to 5% of methyl-β-cyclodextrins comprise 2 methyl groups (DS of 2);
5 to 15% of methyl-β-cyclodextrins comprise 3 methyl groups (DS of 3);
20 to 25% of methyl-β-cyclodextrins comprise 4 methyl groups (DS of 4);
25 to 40% of methyl-β-cyclodextrins comprise 5 methyl groups (DS of 5);
15 to 25% of methyl-β-cyclodextrins comprise 6 methyl groups (DS of 6);
5 to 15% of methyl-β-cyclodextrins comprise 7 methyl groups (DS of 7);
0 to 5% of methyl-β-cyclodextrins comprise 8 methyl groups (DS of 8);
the total sum being generally of the order of 100%, although the composition can optionally contain traces of methyl cyclodextrins of different DS, as well as traces of native, that is non-methylated, cyclodextrin.

Moreover, it is quite possible to consider varying the proportions or to isolate molecules or groups of methyl cyclodextrin molecules, in particular depending on their DS.

Thus, in another particular embodiment, methyl cyclodextrin is a methyl-β-cyclodextrin which exhibits a DS chosen from an integer ranging from 2 to 8, in particular 2, 3, 4, 5, 6, 7 or 8.

In another preferred embodiment, methyl cyclodextrin is a methyl-β-cyclodextrin, at least 50% of the methyl groups being located at the hydroxyl carried by the C2 carbon of the glucopyranose units, preferably between 60 and 80%, typically of the order of 75%, and which has a DS selected from an integer ranging from 2 to 8, in particular 2, 3, 4, 5, 6, 7 or 8.

In another particular embodiment, methyl cyclodextrin, in particular methyl-β-cyclodextrin, has a MS of between 0.1 and 0.3, in particular between 0.2 and 0.3, in particular between 0.20 and 0.30. In another particular embodiment, methyl cyclodextrin, in particular methyl-β-cyclodextrin, has a MS of between 0.3 and 0.5, in particular between 0.30 and 0.50. In another particular embodiment, methyl cyclodextrin, in particular methyl-β-cyclodextrin, has a MS of between 0.5 and 0.6, in particular between 0.50 and 0.60. In another particular embodiment, methyl cyclodextrin, in particular methyl-β-cyclodextrin, has a MS of between 0.6 and 0.7, in particular between 0.60 and 0.70. In another particular embodiment, methyl cyclodextrin, in particular methyl-β-cyclodextrin, has a MS of between 0.7 and 0.8, in particular between 0.70 and 0.80. In another particular embodiment, methyl cyclodextrin, in particular methyl-β-cyclodextrin, has a MS of between 0.8 and 0.9, in particular between 0.80 and 0.90. In another particular embodiment, methyl cyclodextrin, in particular methyl-β-cyclodextrin, has a MS of between 0.9 and 1.1, in particular between 0.90 and 1.10. In another particular embodiment, methyl cyclodextrin, in particular methyl-β-cyclodextrin, has a MS of between 1.1 and 1.2, in particular between 1.10 and 1.20.

Generally, the methyl cyclodextrin used according to the invention has a level of reducing sugars of less than 1% by dry weight, preferentially less than 0.5%.

The composition of methyl-β-cyclodextrins according to the invention can be obtained by the method described in patent U.S. Pat. No. 6,602,860 B1. An example of such a composition is the product KLEPTOSE® CRYSMEB which has a molar substitution value of 0.7 or more precisely 0.67 methylated per unit of glucose.

Optionally, the composition according to the present invention may further comprise a cyclodextrin, in particular β-cyclodextrin, unsubstituted and/or a cyclodextrin, in particular 3-cyclodextrin, substituted with sulfobutylether (SBE-), hydroxyethyl, hydroxypropyl (HP-), carboxymethyl, carboxyethyl, acetyl, triacetyl, succinyl, ethyl, propyl, butyl, sulfate groups, preferably sulfobutyl and hydroxypropyl, preferably with a molar substitution value between 0.05 and 1.5.

However, preferably, the composition of the invention does not comprise other cyclodextrins than methyl cyclodextrin useful for the invention (and optionally native cyclodextrin in trace amounts, as mentioned above).

Optionally, methyl cyclodextrin according to the invention, in particular methyl-β-cyclodextrin, can be substituted by additional groups, in particular chosen from those listed before. It may therefore for example be a sulfated methyl-β-cyclodextrin.

However, preferably, methyl cyclodextrin useful for the invention, in particular methyl-β-cyclodextrin, is not substituted with groups other than methyl groups.

In an alternative embodiment of the invention, the methyl cyclodextrins as defined in the present application and composed of α-D-glucopyranose units connected together by α-1,4 bonds may be partially or entirely substituted by α-D-glucopyranose units connected together by α-1,6 bonds, in the pharmaceutical compositions of the present invention.

In a preferred embodiment of the invention, methyl cyclodextrin is the only active ingredient of the pharmaceutical composition.

In another embodiment, the pharmaceutical composition further comprises one or more active ingredient(s) typically selected from those useful for the prevention and/or treatment of symptoms and/or pathologies associated with hepatic steatosis.

The compositions according to the present invention may also comprise at least one pharmaceutically acceptable excipient. Any excipient suitable for the galenical forms known to a person skilled in the art can be used in particular for systemic administration, preferentially for oral administration of parenteral administration, cutaneous or mucosal administration, in particular subcutaneously, intravenously, intramuscularly, intraperitoneally, nasally, pulmonarily, rectally, dermally, intrathecally or spinally, preferably orally.

Mention may be made, for example, of saline, physiological, isotonic, buffered, etc. solutions, compatible with pharmaceutical use and known to the person skilled in the art. The compositions may contain one or more agents or vehicles selected from dispersants, solubilizers, stabilizers, preservatives, etc. Agents or vehicles that can be used in formulations (liquid and/or injectable) methylcellulose, are in particular hydroxymethylcellulose, carboxymethylcellulose, polysorbate 80, mannitol, gelatin, lactose, plant oils, acacia, liposomes, etc. The compositions can be formulated in the form of injectable suspensions, gels, oils, tablets, suppositories, powders, gelcaps, capsules, aerosols, etc., optionally by means of galenical forms or devices ensuring prolonged and/or delayed release. For this type of formulation, an agent such as cellulose, carbonates or starches is advantageously used.

The compositions capable of being administered by injection in an individual in the context of the invention comprise between 1 and 100 mg/kg, preferentially between 20 and 70 mg/kg, even more preferentially between 30 and 50 mg/kg, and even more preferably 40 mg/kg of methyl cyclodextrin as defined in the present invention, relative to the total weight of the individual. Of course, the person skilled in the art is able to adapt the dose of methyl cyclodextrin defined in the present application according to the weight of the individual to be treated and the mode of administration.

According to a preferred embodiment of the invention, the pharmaceutical composition is capable of being administered in oral form.

When the pharmaceutical composition according to the invention is used in oral form, the amount of methyl cyclodextrin to be administered in an amount capable of reducing the storage of lipids in the liver of the patient. The oral dosage may, for example, be between 10 mg/kg/day and 10,000 mg/kg/day, preferably between 20 mg/kg/day and 7000 mg/kg/day, between 50 mg/kg/day and 5000 mg/kg/day, between 75 mg/kg/day and 4000 mg/kg/day, between 100 mg/kg/day and 3000 mg/kg/day, between 200 mg/kg/day and 2000 mg/kg/day, between 300 mg/kg/day and 1000 mg/kg/day, even more preferably between 400 mg/kg/day and 800 mg/kg/day.

The following examples serve to illustrate and show other aspects and advantages of the invention and must be considered non-limiting.

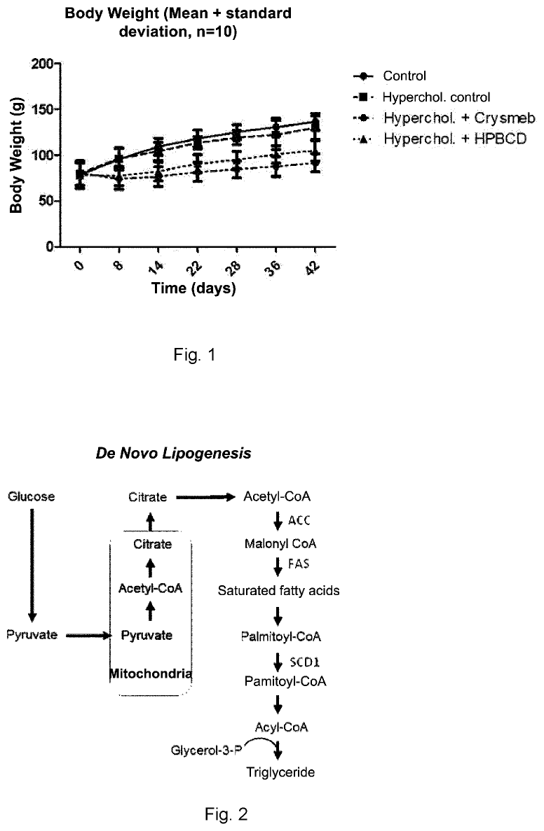

FIG. 1 shows the evolution of the body weight over time of the 4 groups of animals of Example 2

FIG. 2 is a schematic representation of the de novo lipogenesis and of the different genes involved in this biosynthesis route.

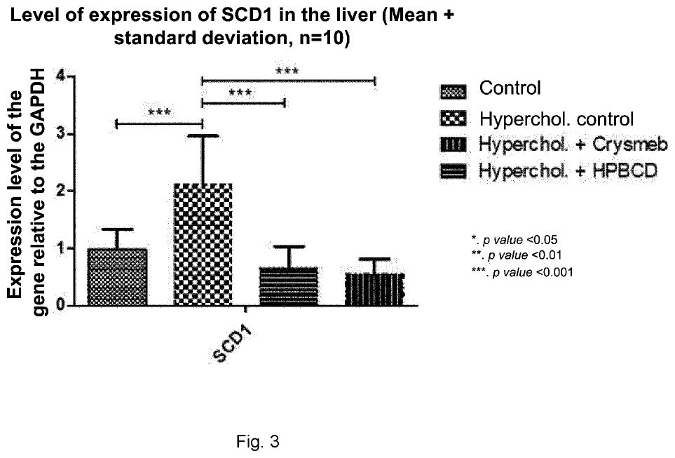

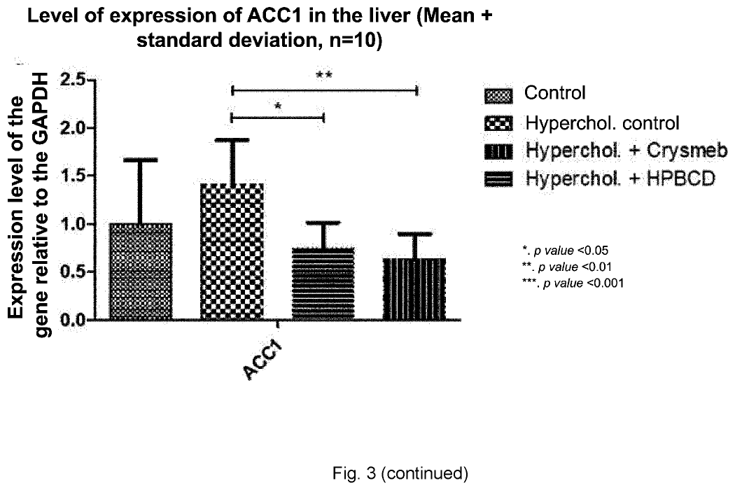

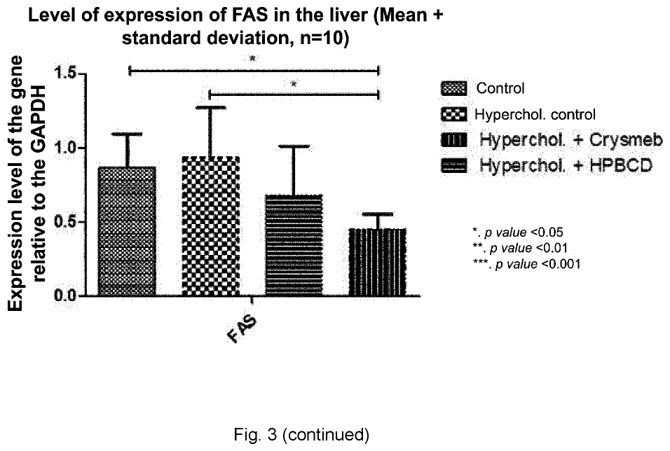

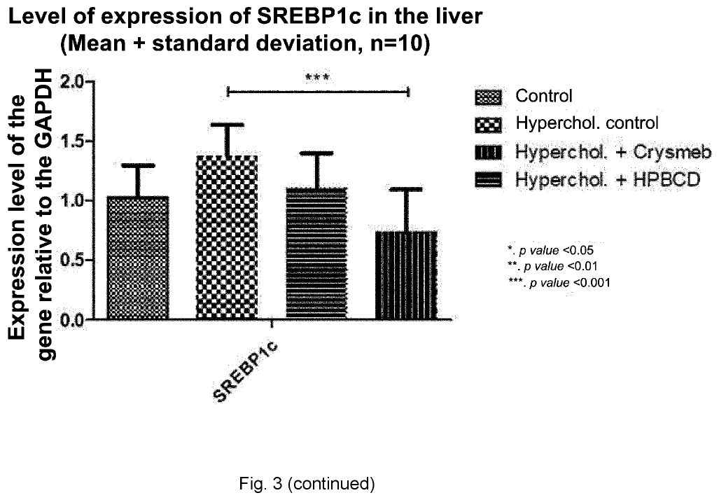

FIG. 3 shows the expression level of different genes involved in de novo lipogenesis in the 4 groups of animals of Example 2

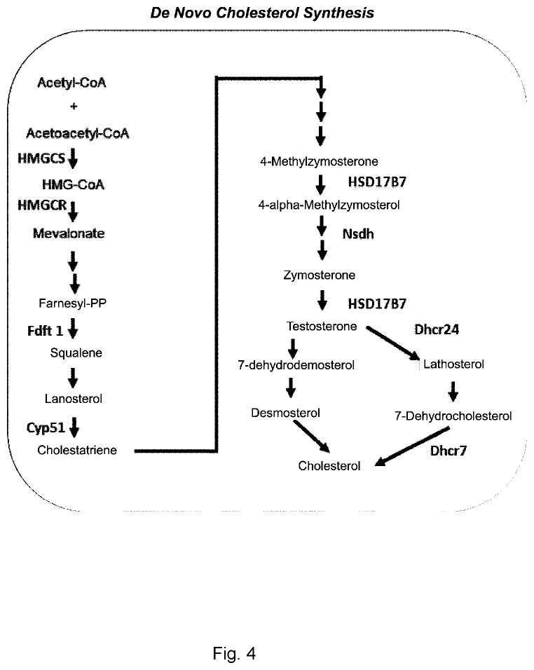

FIG. 4 is a schematic representation of de novo cholesterol synthesis, with the different enzymes involved.

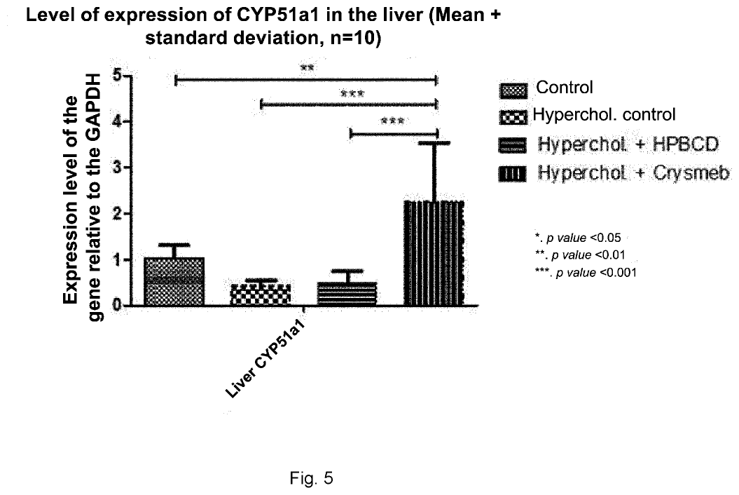

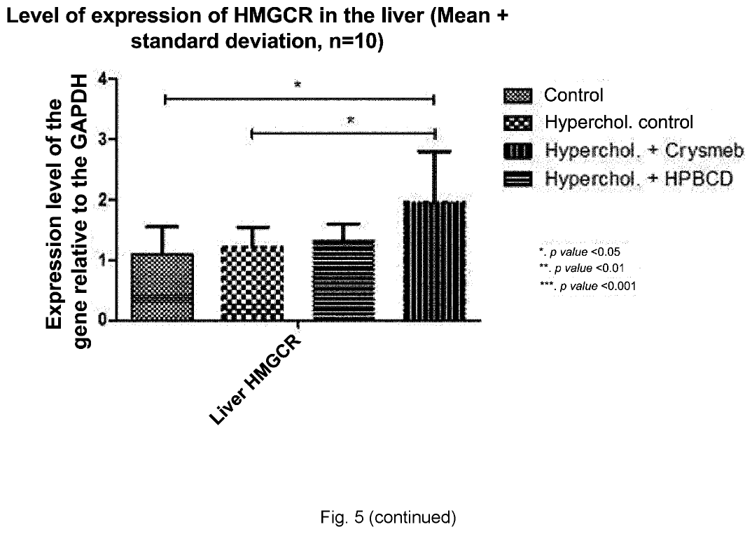

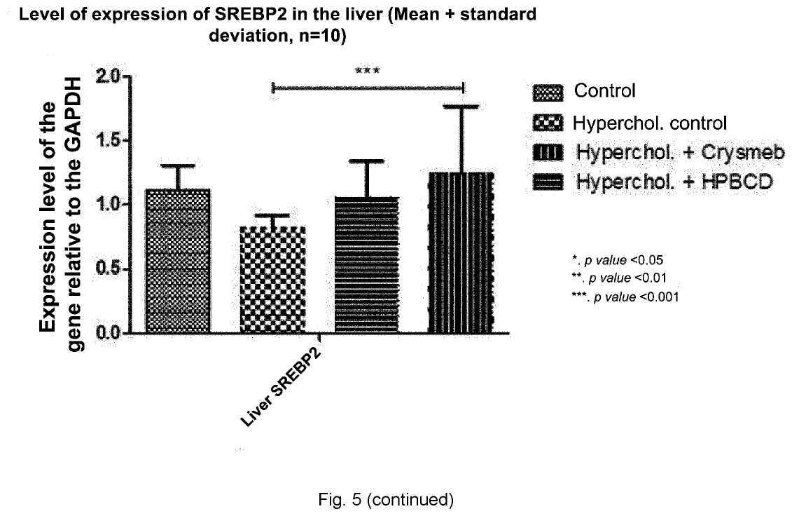

FIG. 5 shows the expression level of different genes involved in de novo cholesterol synthesis, in the 4 groups of animals of Example 2

FIG. 5 shows the expression level of different genes involved in de novo cholesterol synthesis, in the 4 groups of animals of Example 2

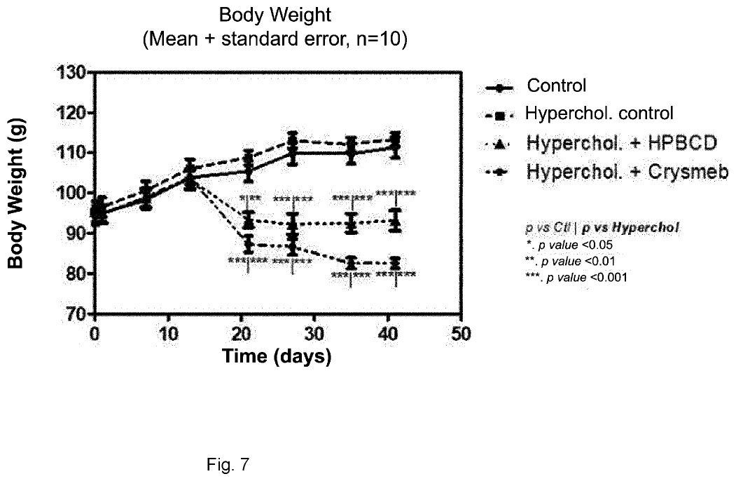

FIG. 7 shows the evolution of the body weight over time of the 4 groups of animals of Example 3

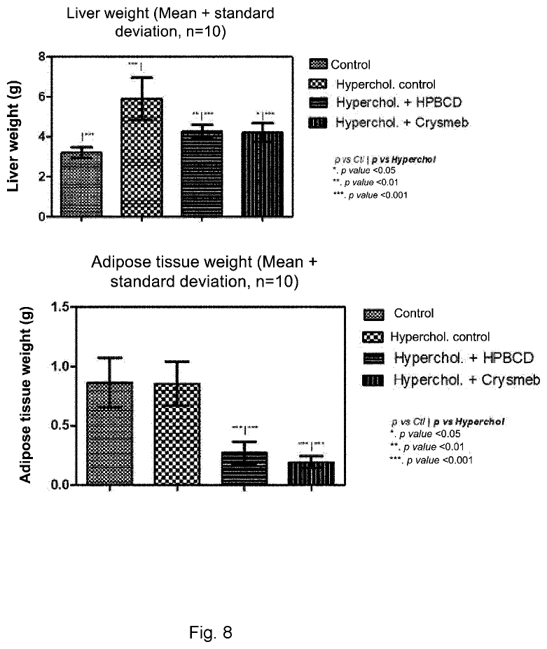

FIG. 8 shows the weight of the liver and of the adipose tissue at the end of the study in the 4 groups of animals of Example 3

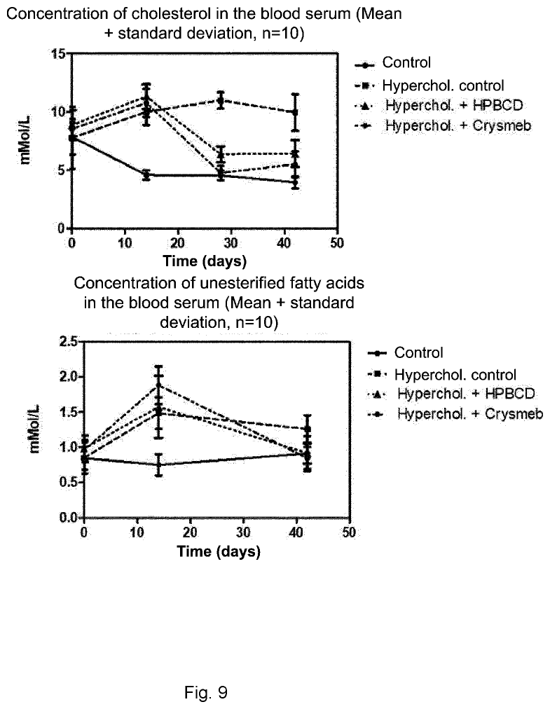

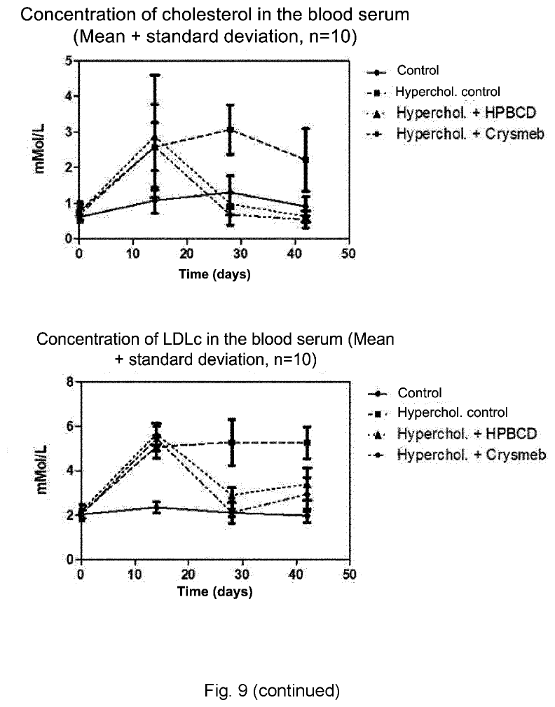

FIG. 9 shows serum cholesterol levels, triglycerides, non-saturated fatty acids and LDLc over time in the 4 animal groups of Example 3.

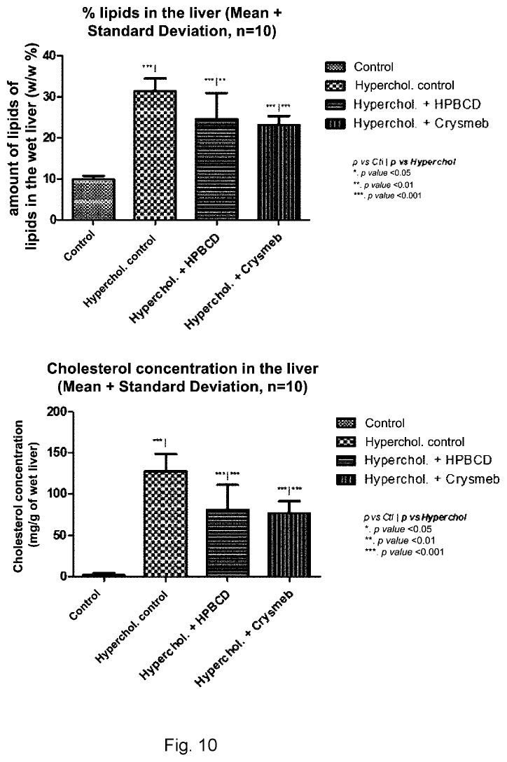

FIG. 10 shows the measurement of the percentage of lipids in the liver and the amount of cholesterol stored in the liver in the 4 groups of animals of Example 3

### EXAMPLES

### Example 1: Materials and Methods

Male LVG golden Syrian hamsters were used for the studies below. The base feed consisted of the food AO4C sold by the company SAFE diet. In the hypercholesterol diets, cholesterol was added (provided by MP Biomedicals)

Different Cyclodextrins were Tested
firstly, hydroxypropyl-β-cyclodextrin (HPBCD) sold by the Applicant under the name “KLEPTOSE® HPB” (oral grade);
secondly, methyl-β-cyclodextrin (MCD) “KLEPTOSE® CRYSMEB”, owned by the Applicant.

### Example 2: Preventive effect of methyl-β-cyclodextrin on hepatic pathologies related to hypercholesterolemia

In this study, 40 Golden Syrian origin LVG hamsters were divided into 4 groups of 10 and subjected for 6 weeks to the following diets:
“Control” group: normal diet
“Control HC” group: hypercholesterol diet (containing 2.5% by weight of cholesterol)
“HC+Crysmeb” group: hypercholesterol diet (containing 2.5% by weight of cholesterol)+3% by weight of Crysmeb (MCD)
“HC+HPBCD” group: hypercholesterol diet (containing 2.5% by weight of cholesterol)+3% by weight of HPBCD.

### Physiological Markers

FIG. 1 shows the evolution of the body weight of the animals during the study. It is found that the hypercholesterol diet did not have any impact on body weight. Conversely, the 2 groups that were supplemented with cyclodextrins had a slower progression.

At the end of 42 days of study, the animals were sacrificed and the weight of the various organs was measured.

Table 1 describes the weight of the aorta, brain, liver, small intestine and adipose tissue of epididymis in the different treatment groups:

TABLE 1

| | | | | | Small | Adipose |
|---|---|---|---|---|---|---|
| | | Aorta | Brain | Liver | intestine | tissue |
| control | N | 10 | 10 | 10 | 10 | 10 |
| | Average | 0.04 | 1.0 | 4.6 | 1.6 | 2.5 |
| | SD | 0.02 | 0.03 | 0.91 | 0.16 | 0.8 |
| HC | N | 10 | 10 | 10 | 10 | 10 |
| | Average | 0.04 | 1.0 | 7.0 | 1.7 | 2.6 |
| | SD | 0.02 | 0.04 | 1.3 | 0.25 | 0.72 |
| | p vs control | — | — | p < 0.001 | — | — |
| HC + | N | 10 | 10 | 10 | 10 | 10 |
| HPBCD | Average | 0.03 | 1.0 | 4.5 | 2.0 | 1.4 |
| | SD | 0.01 | 0.05 | 0.51 | 0.34 | 0.28 |
| | p vs control | — | — | — | p = 0.01 | p < 0.001 |
| | p vs HC | — | — | p < 0.001 | — | p < 0.001 |
| HC + | N | 10 | 10 | 10 | 10 | 10 |
| Crysmeb | Average | 0.02 | 0.9 | 3.4 | 2.0 | 0.9 |
| | SD | 0.01 | 0.03 | 0.33 | 0.29 | 0.23 |
| | p vs control | — | — | p = 0.03 | p = 0.04 | p < 0.001 |
| | p vs HC | p = 0.04 | — | p < 0.001 | — | p < 0.001 |

As shown in Table 1, the tissues that were the most affected by the treatment with cyclodextrins are the tissues involved in the storage of oil: the liver and the adipose tissue of the epididymis (which is strongly correlated with the total adipose mass of an individual). The effect of decreasing the adipose mass was the most marked with the MCD Crysmeb.

Table 2 shows the plasma triglyceride levels in the different groups at different moments of the study: at the beginning (day 0 DO), on day 14 (D14), on day 28 (D28) and at the end of the study (day 43 D43)

TABLE 2

| | | TG D0 | TG D14 | TG D28 | TG D43 |
|---|---|---|---|---|---|
| control | Average | 2.85 | 2.55 | 1.89 | 2.19 |
| | SD | 0.79 | 1.07 | 0.85 | 1.09 |
| HC | Average | 3.41 | 4.37 | 5.19 | 5.01 |
| | SD | 1.10 | 1.58 | 3.19 | 4.10 |
| | p vs | | p < 0.01 | p = 0.002 | |
| | control | | | | |
| HPBCD | Average | 3.03 | 1.28 | 1.23 | 1.58 |
| | SD | 0.71 | 0.41 | 0.19 | 0.65 |
| | p vs | | p = 0.07 | | |
| | control | | | | |
| | p vs HC | | p < 0.001 | p < 0.001 | |
| Crysmeb | Average | 2.88 | 0.89 | 0.64 | 0.54 |
| | SD | 1.42 | 0.48 | 0.15 | 0.12 |
| | p vs | | p = 0.01 | | |
| | control | | | | |
| | p vs HC | | p < 0.001 | p < 0.001 | |

It is found that the HC diet induces a strong increase in the level of triglycerides in the blood, relative to the control group. This increase was normalized by adding cyclodextrins, with a more marked effect for the MCD Crysmeb.

Table 3 provides the biochemical data measured in the liver of the animals at the end of the 42 days of treatment.

TABLE 3

| | | % lipids | | | % lipids | CHOL | TG |
|---|---|---|---|---|---|---|---|
| | | (g/100 g | CHOL | TG | (g of lipid | (mg of chol | (mg of TG |
| | | of liver) | (mg/g | (mg/g | in liver/ | in liver/ | in liver/ |
| | | D0 | liver) | liver) | animal) | animal) | animal) |
| CTRL | Average | 9.90 | 3.54 | 22.26 | 0.43 | 15.04 | 96.99 |
| | SD | 0.79 | 1.87 | 4.55 | 0.06 | 7.60 | 24.55 |
| HC | Average | 28.62 | 103.80 | 37.61 | 2.00 | 713.91 | 253.24 |
| | SD | 4.96 | 14.48 | 11.25 | 0.56 | 115.49 | 66.99 |
| | p vs control | p < 0.001 | p < 0.001 | p = 0.06 | p < 0.001 | p < 0.001 | p < 0.001 |
| HPBCD | Average | 23.02 | 32.98 | 22.73 | 1.04 | 154.19 | 92.12 |
| | SD | 8.62 | 22.84 | 10.13 | 0.38 | 103.36 | 41.19 |
| | p vs control | p < 0.001 | p < 0.001 | | p = 0.01 | p = 0.01 | |
| | p vs HC | | p < 0.001 | p = 0.08 | p < 0.001 | p < 0.001 | p < 0.001 |
| MCD | Average | 11.19 | 6.52 | 26.78 | 0.38 | 22.08 | 94.39 |
| | SD | 1.70 | 3.51 | 16.63 | 0.09 | 12.05 | 70.31 |
| | p vs control | | | | | | |
| | p vs HC | p < 0.001 | p < 0.001 | | p < 0.001 | p < 0.001 | p < 0.001 |

As shown in Table 3, the HC diet induces a strong increase in the amount of lipids stored in the liver, mainly in the form of cholesterol, but also in the form of triglycerides. The addition of cyclodextrin in the feed had the effect of reducing this storage. The effect was even more marked for MCD, for which normal levels of % lipids, storage of cholesterol and triglycerides were measured.

### Biomarkers (Amount of Messenger RNA Expressed in the Liver)

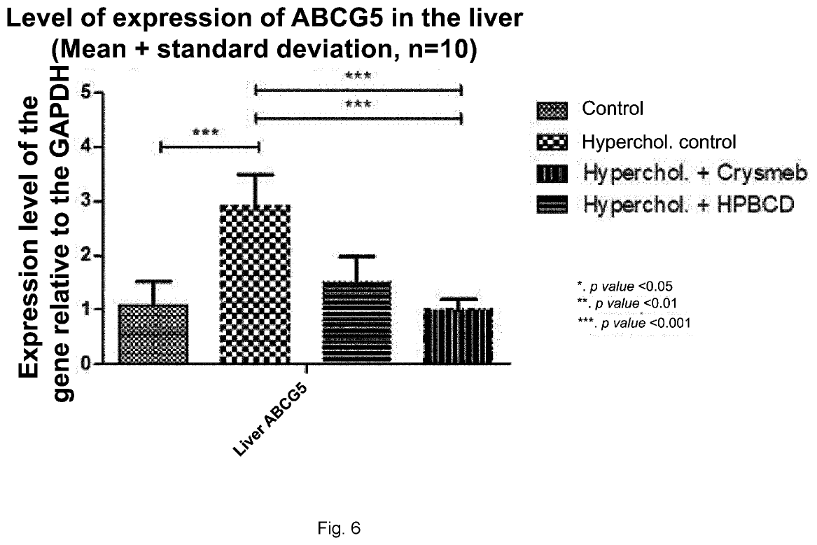

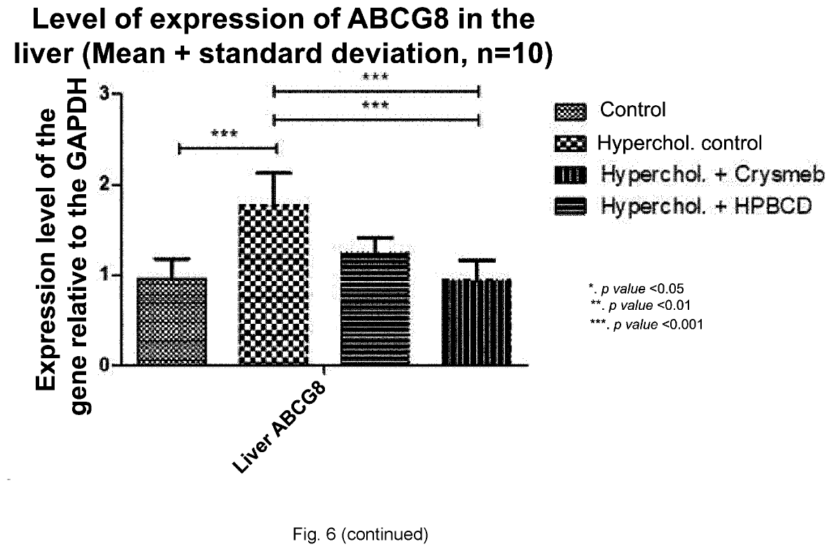

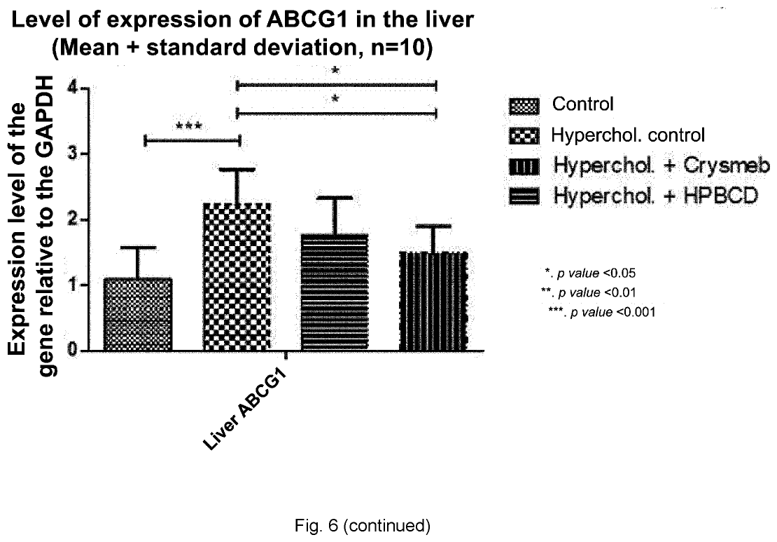

The levels of expression of different genes involved in de novo lipodogenesis (FIG. 3) and in de novo cholesterol synthesis (FIG. 5) and cholesterol removal (FIG. 6) were measured in the liver at the end of the study, in the 4 groups of animals.

The cholesterol-rich diet induced increased expression of SCD1, which must lead to an increase in the production of unsaturated fatty acids, such as triglycerides. The addition of HPBCD in the feed led to an inhibition of the expression of the SCD1 and ACC genes compared to the control group, which could be related to a reduction in the synthesis and storage of fatty acids such as triglycerides. The addition of MCD in the feed led to an even more pronounced effect than the addition of HPBCD and has led to overall inhibition of the expression of the FAS, ACC, SCD1 and SREBP1 genes (FIG. 3).

As shown in FIG. 5, the HC diet leads to a decrease in the expression of the CYP51 gene compared to the control group, and therefore to a reduction in the synthesis of cholesterol. The addition of HPBCD has had little effect. By contrast, the addition of Crysmeb MCB led to an increase in the expression of the SREB2, CYP51a1, and HMGCR genes, and therefore to increased cholesterol synthesis, instead of the synthesis of fatty acids.

As shown in FIG. 6, the addition of MCD normalizes the expression of the genes involved in the removal of cholesterol, relative to the HC group. This normalization is greater with the MCD than with HPBCD.

In conclusion, the various measured parameters demonstrate that the methyl cyclodextrin according to the invention makes it possible to effectively combat disorders due to a hypercholesterol diet (increase in the storage of fatty acids in the liver and increased fatty acids).

The use of methyl cyclodextrin according to the invention promotes a reduction in lipid accumulation in the liver and an increase in the removal of cholesterol.

These effects are greater with the MCD according to the invention, than with another cyclodextrin, the HPBCD.

### Example 3: Curative Effect of Methyl-β-Cyclodextrin on Hepatic Pathologies Related to Hypercholesterolemia

The same protocol as Example 2 was carried out, but with a first phase of induction of hypercholesterolemia for 2 weeks, followed by a second phase of treatment during which the cyclodextrins were added to the supply of the “HC+Crysmeb” and “HC+HPBCD” groups.

In this study, 40 Golden Syrian origin LVG hamsters were divided into 4 groups of 10 and subjected for 6 weeks to the following diets:
“Control” group: normal diet
“Control HC” group: hypercholesterol diet (containing 2.5% by weight of cholesterol)
“HC+Crysmeb” group: hypercholesterol diet (containing 2.5% by weight of cholesterol) from D1 to D14 and then hypercholesterol diet (containing 2.5% by weight of cholesterol)+3% by weight of Crysmeb (MCD) from D15 to D42.
“HC+HPBCD” group: hypercholesterol diet (containing 2.5% by weight of cholesterol) from D1 to D14 and then hypercholesterol diet (containing 2.5% by weight of cholesterol)+3% by weight of HPBCD from D15 to D42.

### Physiological Markers

FIG. 7 shows the evolution of the body weight of the animals during the study. It is found that the hypercholesterol diet did not have any impact on body weight. Conversely, the 2 groups that were supplemented with cyclodextrins had a slower progression.

At the end of 42 days of study, the animals were sacrificed and the weight of the various organs was measured.

FIG. 8, which describes the weight of the liver and the adipose tissue of the epididymis in the different treatment groups, shows that the tissues that were the most affected by treatment by the cyclodextrins are the tissues involved in storing oil: the liver and the adipose tissue of the epididymis (which is strongly correlated with the total adipose mass of an individual). The effect of decreasing the adipose mass was the most marked with the MCD Crysmeb.

FIG. 9 shows serum concentrations of cholesterol, non-saturated fatty acids, triglycerides and LDLc in the different treatment groups over time.

It is found that the HC diet induces a strong increase in the level of triglycerides and cholesterol in the blood, relative to the control group. The addition of the MCD Crysmeb after 14 days of HC diet made it possible to significantly reduce these concentrations, which are closer to those of the control group.

As shown in FIG. 10, the HC diet induces a large increase in the amount of lipids stored in the liver, in particular in the form of cholesterol. The addition of cyclodextrin to the diet after the induction phase had the effect of reducing this storage.

Histologically, the appearance of microvesicular vacuoles and infiltration by inflammatory cells in the HC group were observed. Exposure to MCD made it possible to reduce the severity of these histological signs in the HC+Crysmeb group (data not provided).

In conclusion, the various measured parameters demonstrate that the methyl cyclodextrin according to the invention makes it possible to effectively combat disorders due to a hypercholesterol diet (increase in the storage of fatty acids in the liver and increased fatty acids), even when the MCD is administered after the induction phase of these disorders.

The use of methyl cyclodextrin according to the invention promotes a reduction in lipid accumulation in the liver and an increase in the removal of cholesterol, with a curative effect, in addition to the preventive effect demonstrated in Example 2.

These effects are greater with the MCD according to the invention, than with another cyclodextrin, the HPBCD.

Thus, the Applicant has demonstrated that a methyl cyclodextrin according to the invention was capable of:
reducing the storage of lipids in the body, and in particular in the liver;
increasing metabolism and cholesterol removal;
improving hepatic histology.

These effects were observed when methyl cyclodextrin according to the invention was administered during the induction of hypercholesterolemia (preventive effect model) or after the onset of the induction of hypercholesterolemia (curative effect model).

The effects observed were greater than those observed with another cyclodextrin, hydroxypropyl-β-cyclodextrin.

1. A method for treating and/or preventing hepatic steatosis and a diseases associated with hepatic steatosis, comprising a step of administering an effective amount of a pharmaceutical composition comprising at least one methyl cyclodextrin, wherein the disease associated with hepatic steatosis is selected from the group consisting of acute or chronic liver inflammation, hepatic fibrosis, abdominal obesity, hepatic failure and cirrhosis.

2. The method according to claim 1, wherein the hepatic steatosis is selected from non-alcoholic hepatic steatosis (“non-alcoholic fatty disease” NAFLD) and non-alcoholic steatohepatitis (NASH).

3. The method according to claim 1, wherein the methyl cyclodextrin has a molar substitution value between 0.05 and 1.5.

4. The method according to claim 3, wherein the methyl cyclodextrin has a molar substitution value between 0.2 and 1.2.

5. The method according to claim 3, wherein the methyl cyclodextrin has a molar substitution value between 0.4 and 0.9.

6. The method according to claim 3, wherein the methyl cyclodextrin has a molar substitution value between 0.6 and 0.8.

7. The method according to claim 1, wherein the methyl cyclodextrin is a methyl-β-cyclodextrin.

8. The method according to claim 1, wherein the methyl cyclodextrin is substituted on the hydroxyl carried by the C2 carbon of the glucopyranose units, or by the C3 and/or C6 carbons of the glucopyranose units, or by a combination of the C2, C3 and/or C6 carbons of the glucopyranose units.

9. The method according to claim 7, wherein the methyl cyclodextrin is substituted on the hydroxyl carried by the C2 and C6 carbons of the glucopyranose units.

10. The method according to claim 1, wherein the methyl cyclodextrin composition comprises one or more methyl-3-cyclodextrins chosen from the group consisting of methyl-β-cyclodextrins substituted on the hydroxyl carried by C2 carbon of the glucopyranose units, methyl-β-cyclodextrins substituted on the hydroxyl carried by carbon C3 and/or C6 of the glucopyranose units, methyl-β-cyclodextrins substituted on the hydroxyl carried by carbons C2, C3 and/or C6 of the glucopyranose units and said methyl-β-cyclodextrins having a molar substitution value of between 0.6 and 0.8.

11. The method according to claim 1, wherein the methyl cyclodextrin composition comprises at least 50, 60, or 75% methyls substituted on the hydroxyl carried by the C2 carbon of the glucopyranose units.

12. The method according to claim 1, wherein the composition is capable of being administered orally.

13. The method according to claim 1, further promoting a reduction in lipid storage.

14. The method according to claim 1, further promoting an increase in cholesterol removal.
